(19) United States
(12) Patent Application Publication
(10) Pub. No.: US 2006/0116564 A1
(43) Pub. Date: Jun. 1, 2006

(54) ESOPHAGEAL DIAGNOSTIC SENSOR

(76) Inventors: Martin P. Mintchev, Calgary (CA); Karan V.I.S. Kaler, Calgary (CA); Orly Yadid-Pecht, Calgary (CA)

Correspondence Address: LAW OFFICE OF MARC D. MACHTINGER, LTD., 750 W. LAKE COOK ROAD, SUITE 350, BUFFALO GROVE, IL, 60089, US

(21) Appl. No.: 11/163,342

(22) Filed: Oct. 14, 2005

Related U.S. Application Data
(60) Provisional application No. 60/618,955, filed on Oct. 14, 2004.

(51) Int. Cl. A61B 5/05; A61B 5/103

(52) U.S. Cl. 600/350; 600/547; 600/593

(57) ABSTRACT

Disclosed is an esophageal catheter that is capable of simultaneously measuring impedance, hydrostatic pressure and contact pressure in an esophagus from peristaltic waves, esophageal fluid and the transit bolus in a single test episode. Circumferential impedance sensors include sensing electrodes that are oppositely disposed on the circumferential impedance sensor, and reference electrodes that are also oppositely disposed on the circumferential impedance sensor and interspersed between the sensing electrodes. Accurate impedance measurements can be made in this fashion in a transverse direction in the esophagus. A hydrostatic pressure sensor is disposed at the distal tip of the esophageal probe that has a rigid cover to protect the hydrostatic pressure sensor from contact pressures of the esophagus. In this manner, the hydrostatic pressure sensor can provide purely hydrostatic pressure data from the fluids in the esophagus. Disposed above the hydrostatic pressure sensor, at the distal end of the probe, is an optical contraction sensor that detects both hydrostatic and contact pressure, by detecting the occlusion created by a flexible membrane disposed between an optical source and an optical detector mounted longitudinally in the probe, in response to contractions at the esophagus. The output of the hydrostatic pressure sensor and the optical contraction sensor permits estimations to be made of the contact pressures created by the esophagus.

### CROSS-REFERENCE TO RELATED APPLICATIONS

This application claims the benefit under 35 USC 119 of U.S. provisional application No. 60/618,955 filed Oct. 14, 2004.

### BACKGROUND OF THE INVENTION

The present invention generally pertains to sensors used in the diagnosis of esophageal conditions, including pressure, pH and bolus transit in the esophagus.

Clinical manifestations of esophageal motility disorders include abnormal bolus transit, pH dynamics, pressure changes and reflux of gastric content. Various techniques have been developed to independently monitor pressure changes, gastroesophageal reflux and bolus transit times, including water-perfused and solid-state catheters. In addition, various different transducers are used for measuring pH values in the esophagus: (1) combined glass electrodes; (2) polycrystalline and monocrystalline antimony electrodes; and (3) field-effect transistor electrodes. Multichannel intraluminal impedance and barium radiography are used to study bolus transit times.

Multichannel intraluminal impedance detects bolus transit without using any potentially harmful radiation. The impedance between the longitudinally arranged ring electrodes used by this technique depends on the electrical characteristics of the bolus and changes in the cross-sectional area of the esophageal wall, caused by peristalsis in the presence of bolus.

U.S. Pat. No. 5,833,625 for example discloses an ambulatory reflux monitoring system with pairs of electrodes longitudinally spaced along a catheter, combined with a pressure sensor, for recording and monitoring gastroesophageal reflux.

A particular problem in the design of esophageal diagnostic sensors is to design a sensor that is comfortable for the patient. These sensors may need to be held in the esophagus for long periods of testing. Generally, the smaller the sensor, the better. However, smaller sensors tend to be less discriminating and prone to error, and provide less space for additional sensing devices used to monitor a range of esophageal conditions.

### SUMMARY OF THE INVENTION

According to an aspect of the invention, there is provided a design of an esophageal diagnostic sensor that permits a reduction in size of the sensor. To allow a reduction in size, pairs of electrodes are arranged circumferentially about a sensor body that is typically round or nearly round in section. Each pair of electrodes includes a sensing electrode and a reference electrode. The electrodes may for example be split rings, where a ring is divided into an even number of segments, or may be point electrodes. Wired or wireless communication channels are provided between the electrodes and an external controller.

In another aspect of the invention, there is provided an integrated pressure and impedance catheter capable of simultaneously recording and discriminating between pressure, force, pH and bolus transit phenomena in the esophagus. In another aspect of the invention, there is provided a method for measuring pH and bolus transit in the esophagus comprising: utilizing at least one set of circumferentially arranged electrical impedance electrodes, each set comprising at least one sensing electrode and one reference electrode arranged equidistantly along the circumference of an esophageal probe and isolated by insulating material of the esophageal probe; and detecting impedance measurements generated by the circumferentially arranged electrical impedance electrodes to determine pH and bolus transit in the esophagus.

In another aspect of the invention, there is provided a method for measuring contact and hydrostatic pressure comprising: utilizing an optical contraction sensor having a light source, an optical imager and a flexible membrane disposed between the light source and the optical imager that flexes in response to esophageal contractions and to hydrostatic pressure in the esophagus and occludes the transmission of light between the light source and the optical imager; and generating optical image data from the optical imager that is representative of the contact and hydrostatic pressure applied to the optical contraction sensor.

In another aspect of the invention, there is provided a method of determining an inferior limit of a lower esophageal sphincter in an esophagus comprising: providing an esophageal probe having a hydrostatic sensor located at a distal tip of the esophageal probe and an optical contraction sensor located proximally adjacent to the hydrostatic sensor; inserting the esophageal probe in the esophagus until hydrostatic pressures are sensed by the hydrostatic sensors indicating that the hydrostatic sensor is located at the inferior limit of the lower esophageal sphincter; and detecting a specific pressure signature from the optical contraction sensor that confirms that the optical contraction sensor is located in the lower esophageal sphincter.

In another aspect of the invention, there is provided an integrated esophageal probe that is suitable for ambulatory monitoring and capable of simultaneously measuring impedance, hydrostatic pressure and contact pressure in an esophagus from peristaltic waves, esophageal fluids and transit of bolus in the esophagus in a single test episode comprising: a plurality of circumferential impedance sensors disposed along the length of the esophageal probe that detect impedance in the esophagus that is indicative of pH levels of the fluids in the esophagus and the transit of bolus in the esophagus, the circumferential impedance sensors having at least one sensing electrode disposed on the circumference of the esophageal probe and at least one reference electrode alternately disposed on the circumference of the esophageal probe, and insulators disposed between each electrode; a hydrostatic sensor disposed at a distal end of the esophageal probe that detects esophageal hydrostatic pressure in the esophagus, the hydrostatic sensor having a shield disposed around the hydrostatic sensor to isolate the hydrostatic sensor from esophageal contact pressures; and an optical contraction sensor that detects esophageal contact pressures and esophageal hydrostatic pressure, the optical contraction sensor having a light source, an optical imager and a flexible membrane disposed between the light source and the optical imager that flexes in response to esophageal contractions and occludes the transmission of light between the light source and the optical image.

In another aspect of the invention, there is provided a method of simultaneously monitoring impedance, hydrostatic pressure and contact pressure in an esophagus from peristaltic waves, esophageal fluid and transit of bolus using an esophageal probe is a single test episode comprising: placing a plurality of circumferential impedance sensors along the length of the esophageal probe that detect impedance in the esophagus that is indicative of pH levels of the fluids in the esophagus and the transit of bolus in the esophagus, the circumferential impedance sensors having at least one sensing electrode disposed on the circumference of the esophageal probe and at least one reference electrode alternately disposed on the circumference of the esophageal probe, and insulators disposed between each electrode; placing a hydrostatic sensor at a distal end of the esophageal probe that detects esophageal hydrostatic pressure in the esophagus, the hydrostatic sensor having a shield disposed around the hydrostatic sensor to isolate the hydrostatic sensor from esophageal contact pressure; placing an optical contraction sensor at a distal end of the esophageal probe that detects esophageal contact pressure and esophageal hydrostatic pressure, the optical contraction sensor having a light source, an optical imager and a flexible membrane disposed between the light source and the optical imager that flexes in response to esophageal contractions and occludes the transmission of light between the light source and the optical imager; and using the esophageal hydrostatic pressure detected by the hydrostatic sensor and the esophageal hydrostatic pressure and esophageal contact pressures detected by the contraction sensor to estimate contact pressure in the esophagus. Further aspects of the invention are described in the following.

### BRIEF DESCRIPTION OF THE DRAWINGS

There will now be described preferred embodiments of the invention with reference to the drawings by way of illustration, and without intending to limit the generality of the invention as defined by the claims, in which drawings:

FIG. 1 is an illustration of an embodiment of the present invention, comprising a catheter probe;

FIG. 1A is an illustration of a second embodiment of the invention;

FIG. 2 is an illustration of one embodiment of a circumferential impedance sensor having two sensing and two reference electrodes;

FIGS. 2A and 2B illustrates various embodiments of split ring electrodes;

FIG. 3 is an illustration of one embodiment of the distal end of the probe of FIG. 1;

FIGS. 4A, 4B and 4C illustrate a contraction sensor with a peristalsis wave in different positions;

FIG. 5 is a graph of impedance measurements taken at different frequencies for various values of pH;

FIG. 6 is a table that provides data for pH sensing repeatability;

FIG. 7 is a graph of the impedance changes associated with simulated reflux measurements of two distal impedance electrodes;

FIG. 8 is a graph of the impedance changes associated with simulated bolus transit detected by two distal impedance electrodes; and

FIG. 9 is a table illustrating image attributes at different pressures that are applied to the contraction sensor.

### DETAILED DESCRIPTION OF PREFERRED EMBODIMENTS OF THE INVENTION

In the claims, the word “comprising” is used in its inclusive sense and does not exclude other elements being present. The indefinite article “a” before a claim feature does not exclude more than one of the feature being present. In accordance with the teachings of this patent document, a diagnostic sensor is formed by using one or more sensing devices housed on or in a sensor body. The sensor body may have any size or shape suitable for use in a human esophagus. For example, the cross-sectional shape of the sensor may be round, or nearly round, such as elliptical. In one embodiment, the sensor has the form of a catheter. In another embodiment, the sensor has the form of a pill. The sensor may be cylindrical, spherical, oblate or some other shape with circular symmetry. When the sensor has one dimension longer than another, that dimension defines a longitudinal axis of the sensor. Thus, for a cylindrical catheter, the cylindrical axis is the longitudinal axis of the catheter. For a spherical sensor, the longitudinal axis is any axis. The circumference of a sensor body with a longitudinal axis is a surface of the body extending around the longitudinal axis of the body. In the case of a cylindrical catheter, the circumference is circular. A communication channel is a wired or wireless channel that permits a signal from a sensing device, such as a pressure sensor or electrode, to be communicated outside of the sensor body to a diagnostic instrument, such as a controller or computer. A point electrode is an electrode having a sensing surface whose length and width are approximately equal.

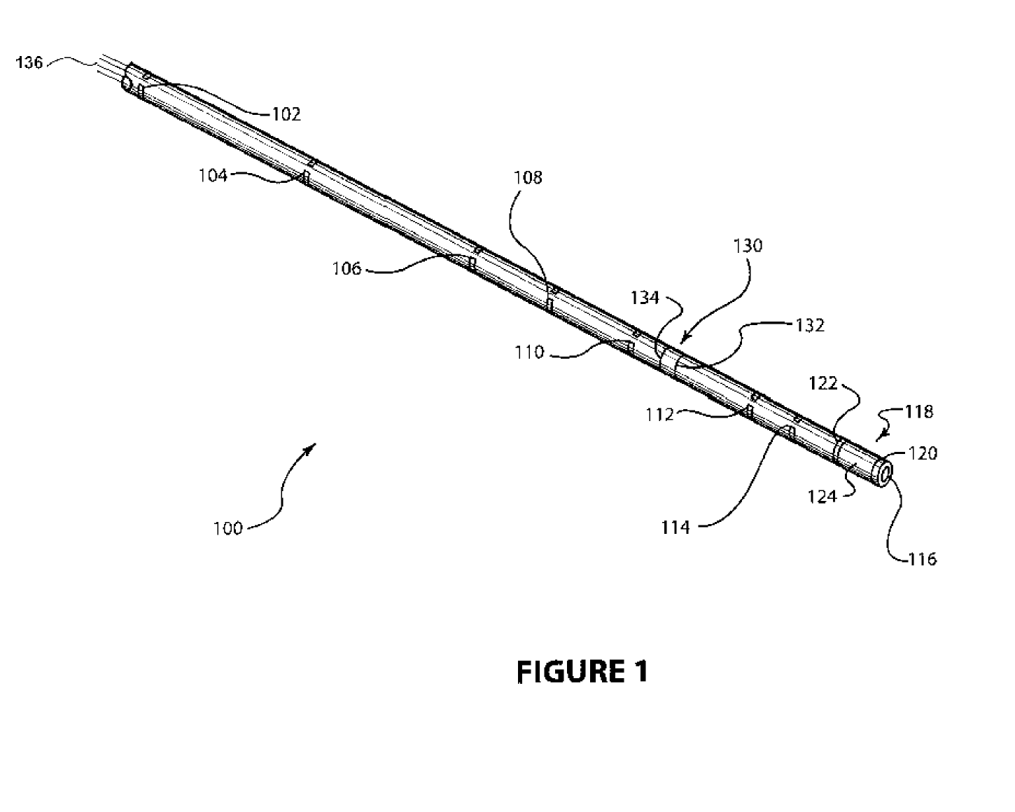

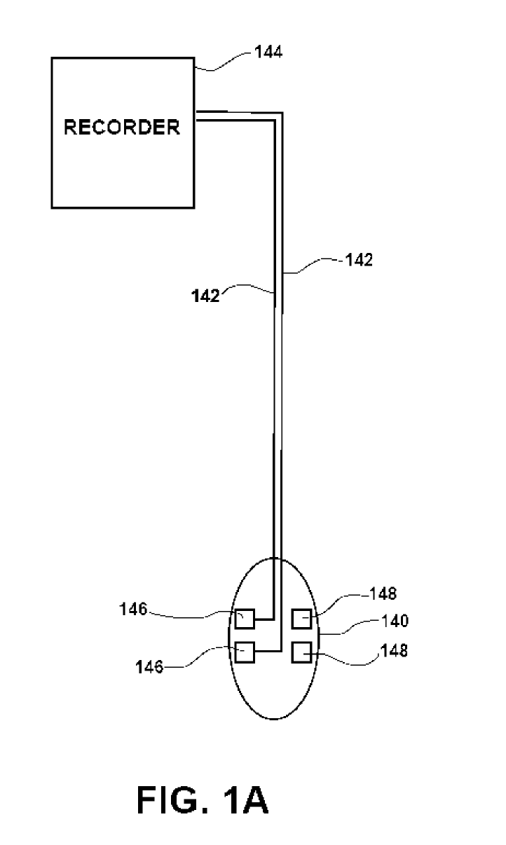

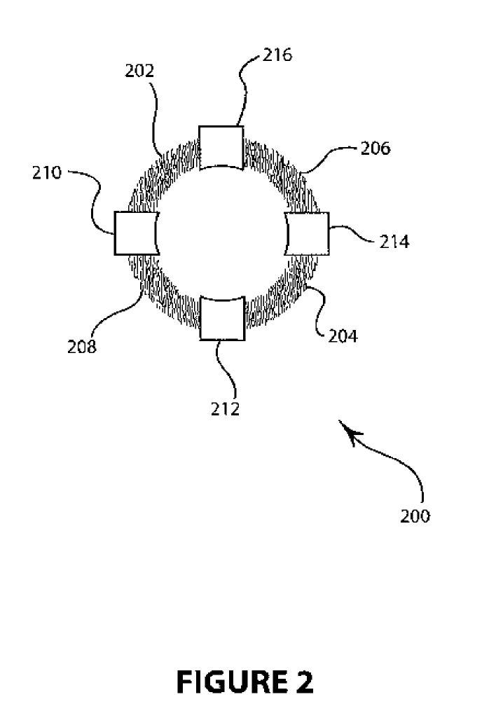

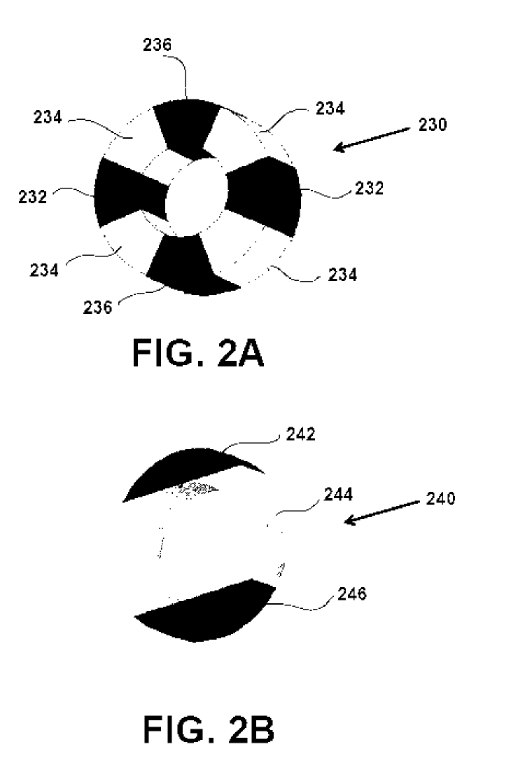

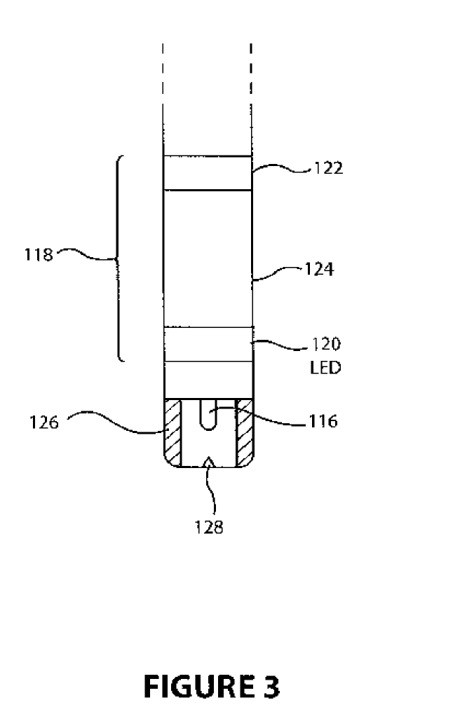

FIG. 1 illustrates one embodiment of an esophageal catheter probe 100 of the present invention. The esophageal catheter probe 100 provides multiple channels of data via communication channels 136 to a recorder for monitoring pH, bolus transit, hydrostatic pressure and contact force, simultaneously and in a single test episode. The esophageal catheter probe 100 includes a plurality of circumferential impedance sensors 102, 104, 106, 108, 110, 112 and 114. The circumferential impedance sensors are disclosed in more detail with respect to FIG. 2. The esophageal catheter probe 100 also includes a hydrostatic pressure sensor 116, that is disclosed in more detail with respect to the description of FIG. 3. The hydrostatic pressure sensor 116 is located on the distal tip of the esophageal catheter probe 100, adjacent to the contraction sensor 118. Contraction sensor 118 includes a light emitting diode 120, or similar light source, a high pressure balloon 124 and an imager 122, such as a CMOS imager that contains multiple pixels.

The circumferential impedance sensors 102-114 are spaced along the length of the esophageal probe 100. In traditional multichannel intraluminal impedance studies, four sensing areas are defined. These areas are located at 5 centimeters, 10 centimeters, 15 centimeters and 20 centimeters above the lower esophageal sphincter (LES). Traditionally, longitudinal, two-ring electrodes are longitudinally placed along the probe to detect impedance values in the esophagus at these locations. The circumferential impedance sensors 102-114 of the catheter are located in the same areas and provide accurate impedance readings, as a result of the use of more than one sensing segment, which avoids possible contact errors related to the internal position of the probe in the esophagus. Again, the circumferential impedance sensors are disclosed in more detail with respect to FIG. 2.

The esophageal catheter probe 100 that is illustrated in FIG. 1 can be constructed of a non-toxic, medical grade material such as polypropylene. Conductors can be molded into, or otherwise disposed in, the interior portion and along the length of the esophageal probe 100 to transmit data from the circumferential impedance sensors 102-114, imager 122 and hydrostatic pressure sensor 116 to the proximal end of the probe 100. In addition, control signals are transmitted through a conductor disposed in the interior portion of the esophageal probe 100 to LED 120.

As disclosed below, the contraction sensor 118 is used to locate the LES with respect to the position of the esophageal probe 100. The contraction sensor 118 provides data that is a combination of contact pressure above the esophageal wall and/or the LES together with hydrostatic pressure of fluids in the esophagus. The hydrostatic pressure sensor 116 provides strictly hydrostatic pressure data. By adaptively compensating the hydrostatic pressure data detected by the hydrostatic pressure sensor 116 from the combined hydrostatic pressure data and contact pressure data detected by the contraction sensor 118, estimates of the contact pressure of the esophageal wall and/or LES can be generated. Adaptive compensation techniques are needed to estimate the contact pressure. Adaptive compensation techniques constitute a traditional method of subtracting signals when the phases of these signals are unknown. Adaptive compensators are disclosed by B. Widrow, M. Lehr, F. Beaufays, E. Wan, M. Bilello, “Adaptive Signal Processing,” World Conference on Neuro Networks '93, Portland, Oreg., July 1993.

As also shown in FIG. 1, a longitudinal impedance sensor 130 is disposed towards the distal end of the esophageal catheter probe 100. The longitudinal impedance sensor 130 comprises a ring 132 and a ring 134. The impedance detected between the rings 132, 134 provides an independent measurement of impedance and, consequently, pH and/or bolus transit. The longitudinal impedance sensor 130 is typically used in multichannel intraluminal impedance devices. The impedance between the longitudinally arranged ring electrodes 132, 134 depends mainly on the change in the cross-sectional area of the esophageal wall caused by peristalsis and bolus transit. In actual esophageal studies, the electrical characteristics of a bolus and the irregular geometry of the esophagus itself affect the impedance readings as well.

FIG. 1A illustrates a further embodiment of the invention in which a diagnostic sensor 140 has the form of a capsule or pill. The diagnostic sensor 140 is connected via wired or wireless communication channels 142 to a recorder 144. A communication channel 142 is provided corresponding to each pair of sensing electrodes, and also, in the case of the pressure sensors in FIG. 1, for each pressure sensor. The communication channel may include multiple wires or frequencies of a radio communication system, but could also include single wires or single frequencies with the data multiplexed onto the communication channels. Recorders for the recording of electrical signals from impedance electrodes and pressure sensors located in the esophagus are known in the art and need not be further described here. An exemplary recorder 144 may use an oscillator to generate a signal to drive the sensing devices, buffer amplifier, isolation amplifier, data acquisition card, filter, processor and graphical user interface, and may be connected to any of various displays or output devices, such as a monitor or printer. Respective pairs of sensing electrodes 146 and reference electrodes 148 are circumferentially spaced around the diagnostic sensor 140. Individual pairs of electrodes are separated from other individual pairs longitudinally. The recorder 144 is used to detect and analyze signals from the sensing electrodes 146 and 148, and in the case of use of pressure sensors illustrated in FIG. 1, also to detect and analyze signals from the pressure sensors.

FIG. 2 discloses one embodiment of a circumferential impedance sensor 200. The circumferential impedance sensor 200 has two sensing electrodes 202, 204 and two reference electrodes 206, 208. The reference electrodes 206, 208 may or may not be internally connected to provide a reference signal. A series of electrical isolators 210, 212, 214 and 216 are disposed between the sensing electrodes 202, 204 and reference electrodes 206, 208. These isolators may simply constitute the material that forms the material of the probe. In addition, as shown in FIG. 2, the sensing electrodes 202, 204 are oppositely disposed on the circumferential impedance sensor 200. Further, the reference electrodes 206, 208 are oppositely disposed on the circumferential impedance sensor 200. The oppositely disposed sensing electrodes 202, 204 and oppositely disposed reference electrodes 206, 208 ensure that proper impedance measurements are taken, since at least one pair of sensor and reference electrodes avoids possible contact errors related to the internal position of the probe in the esophagus. Both the sensing electrodes 202, 204 and reference electrodes 206, 208 may be made from stainless steel or other metal that is impervious to the low pH values of the esophageal fluids to which the esophageal probe 100 is subjected. Alternately, the circumferential impedance sensor 200 can be constructed with one sensing electrode and one reference electrode. These electrodes may be arranged equidistantly around the circumference of the probe 100. Isolators between the electrodes, again, may simply constitute the material that forms the material of the probe. Further, three or more electrodes can also be used depending upon the specific design features of the probe 100.

Examples of split rings are shown in FIGS. 2A and 2B. In FIG. 2A, impedance sensing device 230 comprises two sensing electrodes 232 and two reference electrodes 236 alternating around the impedance sensing device and spaced by insulators 234. In FIG. 2B, impedance sensing device 240 comprises a sensing electrode 242 and a reference electrode 246 circumferentially spaced around the impedance sensing device 240 and spaced by insulators 244.

The concentration of electrolytes in a solution is indicative of both pH and electrical conductivity. During gastro-esophageal reflux, gastric content flows back into the esophagus resulting in a decrease in the esophageal pH. Hence, the impedance measured by the electrodes decreases as well. In traditional multichannel intraluminal impedance, longitudinal impedance recordings are used. The main disadvantage of this technique is that the measurements are affected by the cross-sectional area changes of the esophageal wall, as disclosed above. The circumferentially arranged “broken ring” electrode sensors 102-114 provide impedance measurements that depend mainly on the conductivity of the transversely disposed medium separating them. Changes in the pH of the esophageal fluid result in changes in the detected impedance. An additional advantage of the circumferential electrode arrangement is that more channels can be utilized along the impedance probe in comparison to typical longitudinal electrode sensors, potentially increasing the number of monitoring channels, without necessarily increasing the complexity of the test or the discomfort to the patient. Moreover, the addition of more distal circumferential impedance channels can be utilized to determine the level of the reflux, which are measurements that are nearly impossible to obtain with traditional multichannel intraluminal impedance detectors.

A disadvantage for pressure sensing is the inability to discriminate between hydrostatic pressure and contact force exerted by the esophageal wall during contractions. By preventing the esophageal wall from applying pressure directly on a solid-state transducer, it is possible to sense just the hydrostatic pressure and therefore to discriminate between pressure and contact force. This capability can be achieved using a small pressure transducer is located inside a rigid cover to detect hydrostatic pressures only.

As shown in FIG. 3, the hydrostatic pressure sensor 116 located at the tip on the distal end of the esophageal probe 100, just below the contraction sensor 118. As shown in FIG. 3, the hydrostatic pressure sensor is a small solid-state pressure transducer that is located within the rigid cover 126 so that the hydrostatic pressure sensor 116 is protected from contact pressures created by the esophagus wall and/or LES. As also shown in FIG. 3, the contraction sensor 118 is mounted directly over the hydrostatic pressure sensor 116. The contraction sensor 118 comprises an imager 122, a balloon 124 and a LED 120. Of course, any type of pressure sensor can be used as a hydrostatic pressure sensor 116, including a sensitive strain gauge, for providing strictly hydrostatic pressure sensor readings.

The hydrostatic pressure sensor 116 can be used to position the esophageal probe so that the tip of the esophageal probe is located at the superior limit of the lower esophageal sphincter. The pressure gradients between the esophagus 123 and the stomach (not shown) vary significantly. As the esophageal probe 100 is moved down the esophagus, a change in the hydrostatic pressure is sensed by the hydrostatic pressure sensor 116 that is located on the distal tip of the esophageal probe 100. A probe 100 can then be pulled back until the hydrostatic pressure sensor 116 detects typical esophageal pressures. In this manner, the esophageal probe 100 can be properly located with the hydrostatic pressure sensor 116 at the superior limit of the LES. Alternatively, or in conjunction with the above process, the contraction sensor 118 can be used to assist in accurately positioning the probe 100. The contraction sensor 118, in accordance with one embodiment, has a length of approximately 1 cm to 1.5 cm. When the inferior limit of the LES is identified by the hydrostatic pressure sensor 116, as indicated above, the optical contraction sensor 118 is located in the LES. This fact can be confirmed since the LES has a specific pressure signature which can be identified by the optical contraction sensor. In this manner, the location of the probe 100 can be confirmed by the optical contraction sensor 118. The probe 100 can then be positioned so that the hydrostatic pressure sensor is anchored at the superior limit of the LES, in the manner described above by pulling back the probe 100 until the hydrostatic pressure sensor 116 detects typical esophageal pressures. The optical contraction sensor 118, in this fashion, confirms with a high degree of accuracy both the superior and inferior limit of the LES. Further, circumferential impedance sensor 114, as well as the other impedance sensors are precisely located with respect to the superior limit of the LES.

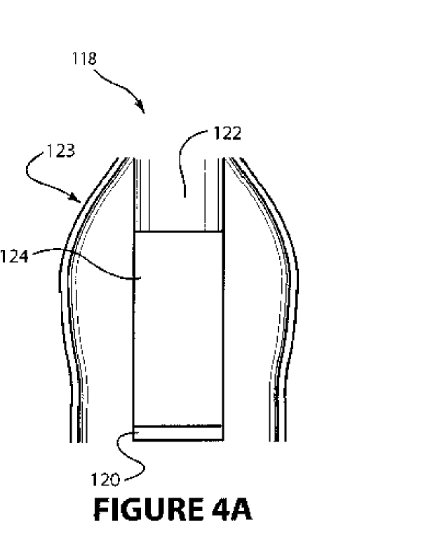

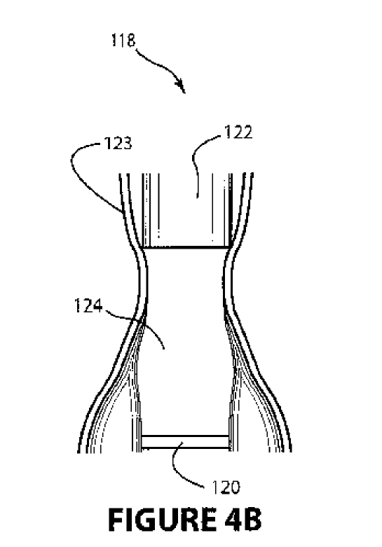

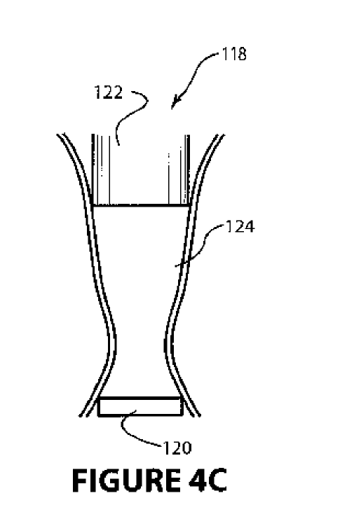

FIGS. 4A, 4B and 4C illustrate the operation of the contraction sensor 118. As illustrated in FIG. 4A, the contraction sensor 118 includes a LED, or other light emitting device 120, an imager such as CMOS imager or other imaging device 122, including other solid-state devices, and a high pressure balloon 124, that is disposed between LED 120 and imager 122. FIG. 4A illustrates the contraction sensor 118 disposed in the esophagus 123. As shown in FIG. 4A, the esophagus is not in a contracted state.

As shown in FIG. 4B, a peristaltic wave in the esophagus 123 has caused the upper portion of the high pressure balloon 124 to be forced inwardly, which reduces the flow of light from LED 120 to imager 122. The contraction of the high pressure balloon 124 partially occludes light from being transmitted from LED 120 to imager 122 as a result of the contraction of the esophagus 123. As a result, the amount of light detected by the imager 122 is reduced by an amount indicative of the amount of contraction of the esophagus 123. In other words, transmitted light manifests itself as an image detected by the imager 122. The dynamics of the modulated intensity of the pixels in the image detected by the imager 122 are representative of the manner in which the peristaltic wave compresses the high blood pressure balloon 124.

FIG. 4C illustrates the movement of the peristaltic wave in a downward direction such that the lower portion of the high pressure balloon 124 is compressed. In other words, the peristaltic wave moves downwardly along the esophagus 123, compressing the high pressure balloon 124 as the wave moves along the contraction sensor 118. The modulation of the intensity of the pixels of the imager 122 provide data regarding both the contact pressure of the esophagus 123, as well as the hydrostatic pressure fluids in the esophagus 123. Since any pressure dynamics between the said light source 120 and the said imager 122 are registered along the entire high pressure balloon 124, optical contraction sensor 118 can be regarded as a new technique to achieve a sleeve type of pressure sensing in the esophagus.

Since the contraction sensor 118 detects both hydrostatic pressure due to fluids in the esophagus 123 and contact pressure of the esophageal wall, contact force can be approximated by subtracting the measured hydrostatic pressure detected by the hydrostatic pressure sensor 116 from the measured readings of the contraction sensor 118.

### EXAMPLES

Eight different impedance channels were monitored from the circumferential impedance sensors 102-114 and the longitudinal impedance sensor 130. The electrodes of sensors 102-114 were driven by an oscillator (Exar, XR-8038, Fremont, Calif.). The resulting current flow was regulated by a potentiometer. The voltage drop between the electrodes of the circumferential impedance sensors 102-114, which is proportional to the electrode impedance, was monitored using a data acquisition system (National Instruments, DAQCard-AI-16XE-50, Austin, Tex.). Hydrochloric acid (0.5 N) was mixed with neutral distilled water in order to test the response of the proposed design to various pH values. The sensing electrodes were submerged in solutions of different pH. Four different pH samples were prepared: 1.4, 2.1, 3.0 and 7.0 using pH/temperature meter (Corning Model 308, Corning Inc., Woburn, Mass.). After each reading the electrodes were cleaned and dried. Two sets of 30 independent measurements were taken at room temperature for each solution of known pH value in order to test repeatability. The voltage drop across the electrodes was measured using a Fluke 87 III True RMS Multimeter (Danaher Corp., Everett, Wash.).

Smashed strawberries were utilized to simulate bolus (pH ranging from 2.3 to 3). Esophageal contractions were simulated using a mechanical model of an esophagus. Bolus transit times were recorded. Reflux periods were simulated and recorded by submerging the probe 100 into a solution with an acidity level similar to the acidity of gastric juice (pH of 1.4). Contractions were simulated by applying pressure around a flexible tube containing a high-pressure balloon and an LED.

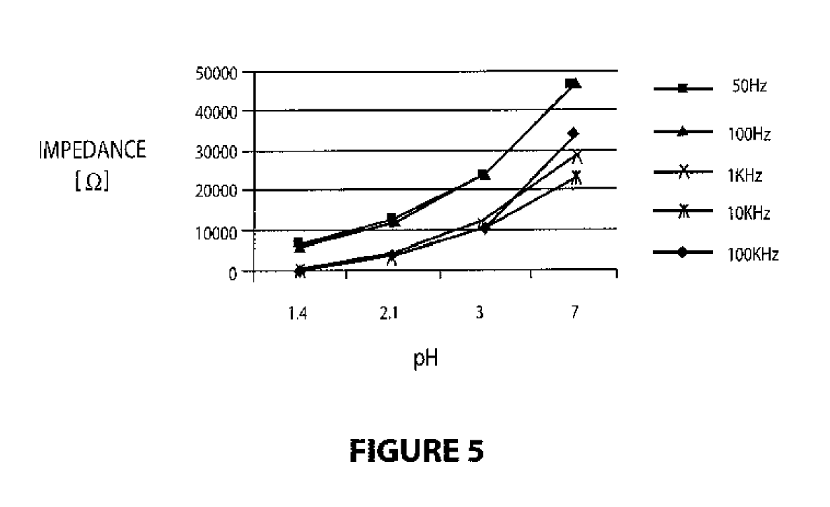

Results: Frequency Selection: A frequency sweep over the frequency range from 50 Hz to 100 KHz was performed in order to monitor voltage changes resulting from changes in the impedance between two circumferentially arranged electrodes submerged in different pH solutions. The solution was modeled as a parallel RC circuit, with impedance given by:

$$Z = \frac{R}{1 + ω 2 R2 C2} - j \frac{ω R2C}{1 + ω 2 R2 C2} \qquad ( 1 )$$

where ω is the radian frequency of the applied AC current, R and C are the lumped equivalent resistance and the capacitance of the bulk solution and the measuring electrodes. From Eq. (1) it can be observed that when a very high frequency is used, the resulting impedance is less representative of the R and C values of the solution and the electrodes. As illustrated in the graph of FIG. 5, 100 Hz the impedance exhibited the largest aperture, ranging approximately between 0 Ω and 50KΩ for the full pH range from 1.4 to 7.

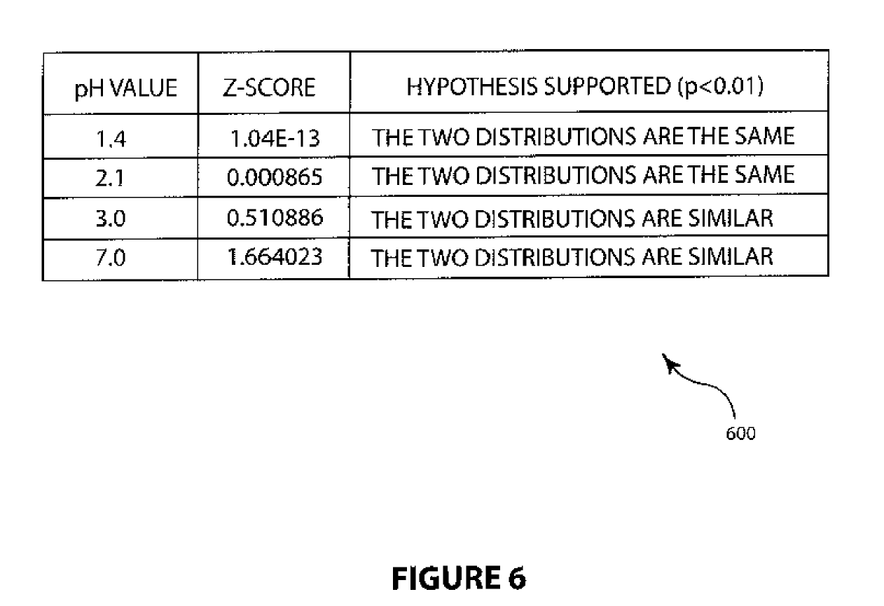

pH Sensing Repeatability Test: The Z-score values calculated for two independent sets of pH measurements are shown in table 600 of FIG. 6. It should be noted that repeatability was particularly good for low pH values, which is important for adequately diagnosing gastro-esophageal reflux.

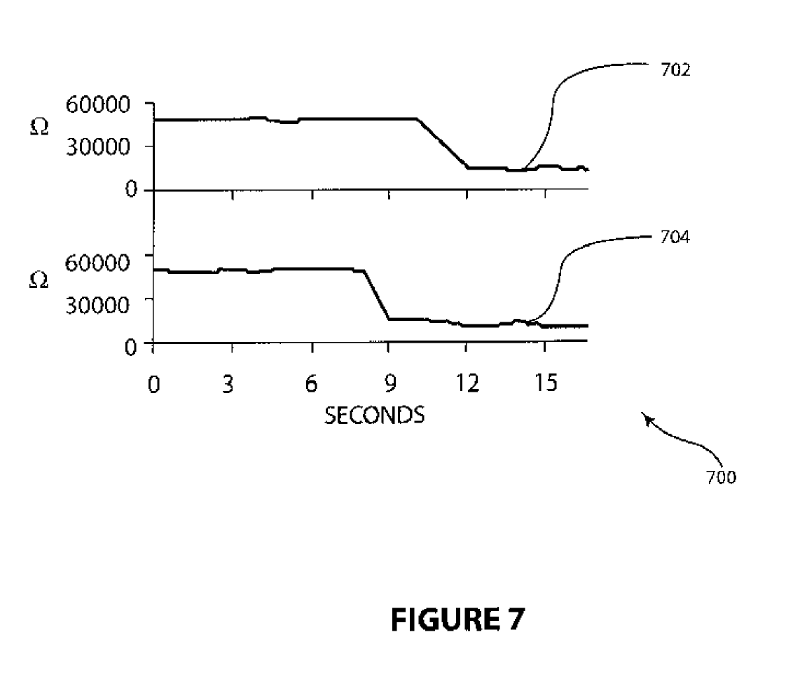

Bolus Transit Time and Reflux Periods Detection: Bolus transit time and reflux periods were recorded using Lab Windows CVI 7.0 (National Instruments, Austin Tex.). FIG. 7 is a graph of impedance changes for the simulated reflux experiment showing the impedance changes over time. Response 702 shows the impedance changes of the circumferential impedance sensor 112. Response 704 shows the impedance changes for circumferential impedance sensor 114. Impedance values in each channel dropped when the bolus or the acid was in contact with sensors 112, 114.

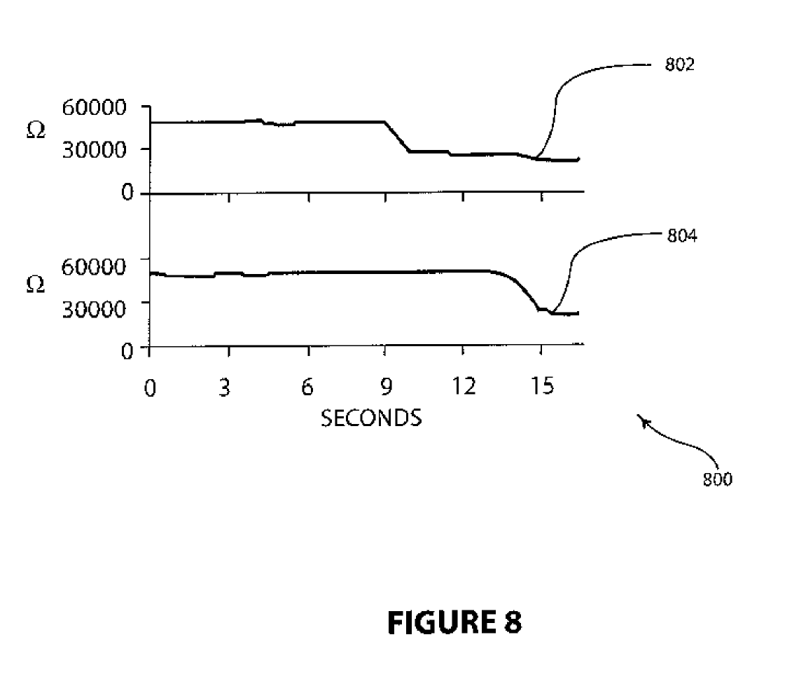

Pressure and Contraction Detection: FIG. 8 is a graph 800 illustrating the impedance changes for the simulated bolus transit experiment. The response 802 illustrates the impedance changes detected by circumferential impedance sensor 112. Response 804 illustrates the impedance changes detected by circumferential impedance sensor 114.

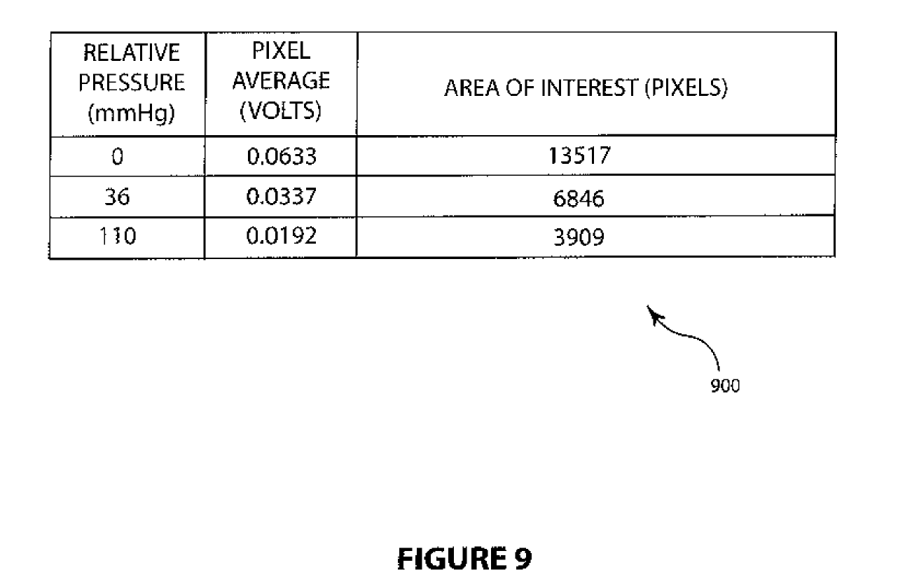

FIG. 9 is a table illustrating the image attributes of the imager 122 with different pressures applied to the contraction sensor 118. The table 900 provides the normalized pixel value averages, approximate diameter of the lighted area on the imager 122 and the areas of interest of the filtered images, i.e., the area in which pixels are above a predetermined threshold, for different pressure values applied to the contraction sensor 118.

The described catheter therefore provides a multichannel esophageal catheter probe that can simultaneously, in one testing session, acquire the desired physical and biophysical quantities of interest in the esophagus. In this fashion, the esophageal catheter probe 100 of the described catheter overcomes disadvantages of existing esophageal catheters. In addition, the compact design of the esophageal probe 100 allows the probe 100 to be used for ambulatory monitoring with minimal discomfort to the patient. The circumferential electrode sensor arrangement minimizes the effective changes in the cross-sectional area of the esophagus wall which influences longitudinal impedance sensors. Accurate and reliable impedance readings were obtained in experimental results using low frequencies of approximately 100 Hz using the parallel RC circuit model. The circumferential impedance sensors 102-114 were able to accurately discriminate between time intervals in which the pH was higher or lower than 4, which satisfied the DeMeester and Johnson scoring system. The design of the circumferential impedance sensors 102-114 allows the length of the probe to be utilized more efficiently and the addition of more impedance monitoring channels. By adding more circumferential impedance sensors, additional impedance readings can be made to more reliably detect the reflux level in the esophagus. Further, the use of a hydrostatic pressure sensor and a contraction sensor allows the operator to discriminate between hydrostatic pressure and contact pressure inside the esophagus. Since good correlation between the image attributes detected by contraction sensor 118 and the pressure applied to the contraction sensor 118 were obtained, a clear avenue is provided by the described catheter for distinguishing between hydrostatic pressure and contact force phenomena in the esophagus. In other words, since the hydrostatic pressure sensor 116 detects only hydrostatic pressure, and the contraction sensor 118 detects both contact pressure and hydrostatic pressure, estimations can be made of the contact pressure by adaptively compensating the hydrostatic pressure readings from the combined contact and hydrostatic pressure readings. Image processing techniques were used in the experimental testing to confirm a good correlation between the image attributes and the pressures applied to the contraction sensor 118. The use of equidistantly positioned alternating sensor electrodes and opposing reference electrodes in the circumferential impedance sensors ensured accurate and reliable impedance measurements in experimental tests.

The foregoing description of the invention has been presented for purposes of illustration and description. It is not intended to be exhaustive or to limit the invention to the precise form disclosed, and other modifications and variations may be possible in light of the above teachings. The embodiment was chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

What is claimed is:

1. An esophageal diagnostic sensor, comprising:
a sensor body having a longitudinal axis and a circumference, the sensor body having a size and shape suitable for use within the esophagus of a human;
a sensing electrode and a reference electrode arranged circumferentially about the sensor body, the sensing electrode and reference electrode defining a first pair of electrodes; and
a communication channel connected to the first pair of electrodes.

2. The esophageal diagnostic sensor of claim 1 in which the sensor body is round in section.

3. The esophageal diagnostic sensor of claim 1 further comprising:
a second sensing electrode and second reference electrode arranged circumferentially about the sensor body, the second sensing electrode and second reference electrode defining a second pair of electrodes; and
a second communication channel connected to the second pair of electrodes.

4. The esophageal diagnostic sensor of claim 3 in which the first pair of electrodes and second pair of electrodes are located at the same position longitudinally on the sensor body.

5. The esophageal diagnostic sensor of claim 3 in which the first pair of electrodes and second pair of electrodes are longitudinally spaced from each other on the sensor body.

6. The esophageal diagnostic sensor of claim 1 further comprising:
multiple pairs of electrodes located circumferentially on the sensor body, at least two or more of the multiple pairs of electrodes being spaced at different longitudinal positions on the sensor body; and
a corresponding communication channel connected to each respective pair of electrodes.

7. The esophageal diagnostic sensor of claim 1 in which each of the reference electrode and the sensor electrode is a point electrode.

8. The esophageal diagnostic sensor of claim 1 in which each of the reference electrode and the sensor electrode is part of a split-ring.

9. The esophageal diagnostic sensor of claim 1 in which the sensor body houses a pressure sensor.

10. The esophageal diagnostic sensor of claim 9 in which the pressure sensor comprises an optical contraction sensor having a light source, an optical imager and a flexible membrane disposed between the light source and the optical imager that flexes in response to esophageal contractions and to hydrostatic pressure in the esophagus and occludes the transmission of light between the light source and the optical imager.

11. The esophageal diagnostic sensor of claim 10 in which the sensor body further houses a hydrostatic pressure sensor.

12. The esophageal diagnostic sensor of claim 9 in which the sensor body is a catheter.

13. The esophageal diagnostic sensor of claim 1 in which the sensor body is a capsule.

14. A method of sensing one or more conditions of the esophagus, the method comprising the steps of:
placing a sensor body in the esophagus, where the sensor body has a longitudinal axis and a circumference, with a sensing electrode and a reference electrode arranged circumferentially about the sensor body, the sensing electrode and reference electrode defining a first pair of electrodes; and
detecting and analyzing signals sent from the first pair of electrodes along a communication channel connected to the first pair of electrodes.

15. The method of claim 14 further comprising detecting signals sent along a communication channel from a second sensing electrode and second reference electrode arranged circumferentially about the sensor body, the second sensing electrode and second reference electrode defining a second pair of electrodes.

16. The method of claim 15 in which the first pair of electrodes and second pair of electrodes are located at the same position longitudinally on the sensor body.

17. The method of claim 15 in which the first pair of electrodes and second pair of electrodes are longitudinally spaced from each other on the sensor body.

18. The method of claim 14 further comprising detecting signals sent along mutliple communication channels from multiple pairs of electrodes arranged circumferentially about the sensor body.

19. The method of claim 1 further comprising detecting and analyzing signals from a pressure sensor on the sensor body.

20. The method of claim 19 further comprising sensing hydrostatic pressure in the esophagus.

21. The method of claim 20 further comprising using the pressure sensor and hydrostatic pressure sensor to distinguish pressure due to contraction of the esophagus.

22. An integrated esophageal probe that is suitable for ambulatory monitoring and capable of simultaneously measuring impedance, hydrostatic pressure and contact pressure in an esophagus from peristaltic waves, esophageal fluids and transit of bolus in the esophagus in a single test episode comprising:
a plurality of circumferential impedance sensors disposed along the length of the esophageal probe that detect impedance in the esophagus that is indicative of pH levels of the fluids in the esophagus and the transit of bolus in the esophagus;
the circumferential impedance sensors having at least one sensing electrode disposed on the circumference of the esophageal probe and at least one reference electrode alternately disposed on the circumference of the esophageal probe, and insulators disposed between each electrode;
a hydrostatic sensor disposed at a distal end of the esophageal probe that detects esophageal hydrostatic pressure in the esophagus, the hydrostatic sensor having a shield disposed around the hydrostatic sensor to isolate the hydrostatic sensor from esophageal contact pressures;
and an optical contraction sensor that detects esophageal contact pressures and esophageal hydrostatic pressure, the optical contraction sensor having a light source, an optical imager and a flexible membrane disposed between the light source and the optical imager that flexes in response to esophageal contractions and occludes the transmission of light between the light source and the optical image.

23. A method of simultaneously monitoring impedance, hydrostatic pressure and contact pressure in an esophagus from peristaltic waves, esophageal fluid and transit of bolus using an esophageal probe is a single test episode, the method comprising the steps of:
placing a plurality of circumferential impedance sensors along the length of the esophageal probe that detect impedance in the esophagus that is indicative of pH levels of the fluids in the esophagus and the transit of bolus in the esophagus, the circumferential impedance sensors having at least one sensing electrode disposed on the circumference of the esophageal probe and at least one reference electrode alternately disposed on the circumference of the esophageal probe, and insulators disposed between each electrode;
placing a hydrostatic sensor at a distal end of the esophageal probe that detects esophageal hydrostatic pressure in the esophagus, the hydrostatic sensor having a shield disposed around the hydrostatic sensor to isolate the hydrostatic sensor from esophageal contact pressure;
placing an optical contraction sensor at a distal end of the esophageal probe that detects esophageal contact pressure and esophageal hydrostatic pressure, the optical contraction sensor having a light source, an optical imager and a flexible membrane disposed between the light source and the optical imager that flexes in response to esophageal contractions and occludes the transmission of light between the light source and the optical imager;
and using the esophageal hydrostatic pressure detected by the hydrostatic sensor and the esophageal hydrostatic pressure and esophageal contact pressures detected by the contraction sensor to estimate contact pressure in the esophagus.
